(19) United States
(12) Patent Application Publication
(10) Pub. No.: US 2020/0069758 A1
(43) Pub. Date: Mar. 5, 2020

(54) COMPOSITION FOR USE IN THE PREVENTIVE AND/OR CURATIVE TREATMENT OF NON-ALCOHOLIC FATTY LIVER DISEASE

(71) Applicant: NORMAPHAR SA, Lasne (BE)

(72) Inventors: Guy WYVEKENS, Lasne (BE); François MOTTE, Lasne (BE); Coralie BRANCART, Rosières (BE)

(21) Appl. No.: 16/119,082

(22) Filed: Aug. 31, 2018

(51) Int. Cl. A61K 36/48; A61K 31/357; A61K 36/9066; A61K 9/00; A61K 45/06; A61P 1/16

(52) U.S. Cl. CPC A61K 36/48; A61K 31/357; A61K 36/9066; A61K 47/38; A61K 45/06; A61P 1/16; A61K 9/0053

(57) ABSTRACT

A composition for use in the preventive and/or curative treatment of non-alcoholic fatty liver disease, in particular for use in the preventive and/or curative treatment of simple fatty liver disease and/or non-alcoholic steatohepatitis.

### FIELD OF THE INVENTION

This invention provides a composition for use in the preventive and/or curative treatment of non-alcoholic fatty liver disease, in particular intended for use in the preventive and/or curative treatment of simple fatty liver disease and/or non-alcoholic steatohepatitis.

### BACKGROUND

Non-alcoholic fatty liver disease (NAFLD) is a very common cause of chronic liver disease, given the rising prevalence of overweight and obesity across the world. Usually affecting patients suffering from metabolic syndrome unrelated to excessive alcohol consumption, non-alcoholic fatty liver disease is considered as one of the main manifestations of said metabolic syndrome. More particularly, non-alcoholic fatty liver disease covers two forms: one form is simple fatty liver disease and the second is non-alcoholic steatohepatitis (NASH or more commonly “non-alcoholic fatty liver”).

Simple fatty liver disease is characterised by steatosis exclusively involving liver tissue (build-up of lipids, mainly triglycerides, in the liver and hepatic cells) associated with fatty degeneration of over 5% of the hepatic cells without either inflammation or fibrosis. In contrast, NASH corresponds to fatty liver disease with inflammation of the liver tissue, with or without fibrosis. Since these two pathologies are relatively close to one another, only histological analysis can differentiate between simple fatty liver disease and NASH.

Using markers for fibrosis in the blood, it can be reliably determined whether or not someone is suffering from non-alcoholic fatty liver disease. In particular, distinction is made between:
indirect markers like transaminases, e.g. aspartate aminotransferase (AST) and alanine aminotransferase (ALT); and
direct markers like type III procollagen, hyaluronic acid, metalloproteases (TIMP-1, TIMP-2, MMP-2, etc.) and cytokines (TGFβ, IL-10, etc.).

Direct markers are constituents of the extracellular matrix or proteins that can be assayed in the blood and, as with indirect markers, knowing their concentrations makes it possible to determine whether or not a subject is suffering from non-alcoholic fatty liver disease.

These days, as mentioned above, the prevalence of these two forms of non-alcoholic fatty liver disease is on the rise across the world, mainly because of the rising prevalence of metabolic syndrome (which corresponds to a combination of obesity, hypertension, hypertriglyceridaemia, low HDL-cholesterol and glucose intolerance or full-blown type 2 diabetes). In the United States for example, the prevalence of non-alcoholic fatty liver disease is estimated at between 20% and 30% of the population, and that of NASH at 5%. It is noted that simple fatty liver disease and NASH are two times more frequent in patients with metabolic syndrome and that risk factors include type 2 diabetes, a family history, overweight (a Body Mass Index of over 25 kg/m²), hypertriglyceridaemia and hypertension.

Nevertheless, one of the risk factors most commonly highlighted is insulin resistance, a pathology observed in overweight patients leading to very high levels of insulin in the blood (hyperinsulinaemia) in an attempt to reduce the blood glucose concentration. One consequence of insulin resistance is stimulation of lipolytic activity in abdominal adipose tissue and the release of free fatty acids which then travel to the liver via the portal vein. This lipolytic activity is largely responsible for the damage seen in non-alcoholic fatty liver disease which itself exacerbates insulin resistance by stimulating the production of glucose in the liver: the result is reactional hyperinsulinism.

In parallel to metabolic syndrome, a chronic pro-inflammatory state is seen as manifested by the presence of inflammatory cells (macrophages and lymphocytes) in visceral adipose tissue and increased production of a range of inflammatory cytokines (including IL6, TNF-α and IL8) that travel to the liver via the portal vein and thence into the blood stream. Build-up of fatty acids in the liver and this chronic pro-inflammatory state probably help favour the oxidative stress that plays a central role in causing the damage of non-alcoholic fatty liver disease.

The classic model for the onset of simple fatty liver disease and NASH involves fatty degeneration of hepatic cells in the liver and also inflammation and fibrosis, notably due to oxidative stress and the presence of cytokines. Recent studies show that build-up of lipids in the liver is mainly due to massive import of free fatty acids, variously due to heavy dietary intake, the release of free fatty acids from adipose tissue and de novo free fatty acid synthesis. It seems that mitochondrial oxidative stress, stressing of the endoplasmic reticulum and cytokines can also lead to fatty degeneration of hepatic cells and thereby cause non-alcoholic fatty liver disease.

Simple fatty liver disease, characterised exclusively by fatty degeneration of hepatic cells, is considered to be a benign form that does not affect life expectancy whereas NASH is considered as a more advanced form since 10-15% of NASH patients also present with cirrhosis of the liver and fibrosis, and hepatocellular carcinoma can develop if the disease progresses. It is also noted that the risk of developing other manifestations of metabolic syndrome is considerably higher in patients with simple fatty liver disease and/or NASH, e.g. the risk of diabetes is 2.5-fold higher. Moreover, it has been demonstrated that simple fatty liver disease and NASH are risk factors for cardiovascular morbidity and mortality: the risk of dying from cardiovascular disease is twice as high in patients with simple fatty liver disease and/or NASH.

To date, there is no reliably established pharmacological treatment for simple fatty liver disease and NASH. For this reason, standard treatment until now has depended upon sustainably reducing body weight through dieting, exercise and regular physical activity in everyday life; in other words, lifestyle changes. The effectiveness of this regimen, as measured by normalisation of transaminase levels and reduction of the steatosis and inflammation, has been demonstrated in various studies but, unfortunately, many patients fail to meet therapeutic targets. In practice, only 15% of patients manage to lose over 10% of their body weight and most of these put the weight back on again within months.

Several pharmacological approaches to treating non-alcoholic fatty liver disease have also been tried. Agents that tackle insulin resistance have been focused on but results have been disappointing. Drugs like metformin and rosiglitazone have been tested but they failed to significantly reduce non-alcoholic fatty acid disease.

In contrast, pioglitazone seems to enhance sensitivity to insulin and reduce simple fatty liver disease and NASH. However, long-term administration can entail certain complications (heart failure, osteoporosis and weight gain) which limits the drug's usefulness.

High-dose Vitamin E has also been studied for administration to patients with non-alcoholic fatty liver disease who do not suffer from diabetes: reductions were observed in the inflammation and non-alcoholic fatty liver disease but high-dose Vitamin E increases all-cause mortality which necessarily limits its use.

Other treatment modalities based on plant extracts have also been attempted but these have been only moderately or even negligibly effective to date. For example, a composition containing an extract of Silybum marinum with an antioxidant, in particular the antioxidant superoxide dismutase (SOD), is known to the background art for treatment of non-alcoholic fatty liver disease. Outcomes with such a composition are satisfactory but nevertheless limited.

Currently, there is unfortunately no medicinal treatment offering a suitable composition which is safe and effective for the preventive and/or curative treatment of non-alcoholic fatty liver disease, in particular for the preventive and/or curative treatment of simple fatty liver disease and/or non-alcoholic steatohepatitis.

There exists therefore a real need to develop and formulate a composition for the preventive and/or curative treatment of non-alcoholic fatty liver disease, said composition being both devoid of danger to health and effective for such treatment.

### SUMMARY

The purpose of the invention is to remedy the deficiencies of the background art by providing such a composition as indicated at the beginning, said composition comprising:
at least a plant extract from the genus Desmodium as antioxidant, and
at least a flavonolignan-type compound and/or at least a compound comprising at least a flavonolignan-type molecule.

In the sense of this invention, the term “plant extract” means any form that makes it possible to use plant-derived active substances obtained from any part of the plant (flower, bark, root, fruit, etc.). Preferably according to the invention, the plant extract is in powder form.

Flavonolignans are polyphenol-type compounds constituted by a flavonoid and a lignan. More specifically, flavonoids are secondary metabolites in plants with a common basic structure formed of two aromatic rings joined by three carbon atoms, and lignan is a phenolic compound formed of two monolignol units.

In the framework of this invention, it was surprisingly determined that such a composition comprising at least a plant extract from the genus Desmodium as antioxidant and at least a flavonolignan-type compound and/or at least a compound comprising at least a flavonolignan-type molecule, allows significantly more effective preventive and/or curative treatment of both forms of non-alcoholic fatty liver disease (simple fatty liver disease and NASH) with no negative impact on patient health, said at least a plant extract from the genus Desmodium as antioxidant acting synergistically with said at least a flavonolignan-type compound and/or said at least a compound comprising at least a flavonolignan-type molecule.

Advantageously in a composition according to the invention, said at least a plant extract from the genus Desmodium as antioxidant is present at a concentration of 5-55% by weight compared to the total weight of the composition, preferably at a concentration of 10-30% by weight compared to the total weight of the composition.

Preferably in a composition according to the invention, said at least a flavonolignan-type compound is present at a concentration of 5-90% by weight compared to the total weight of the composition, preferably at a concentration of 15-30% by weight compared to the total weight of the composition.

Preferentially in a composition according to the invention, said at least a compound comprising at least a flavonolignan-type molecule is present at a concentration of 5-90% by weight compared to the total weight of the composition, preferably at a concentration of 15-30% by weight compared to the total weight of the composition.

It has been determined that such proportions (quantities by weight) of the various ingredients of a composition according to the invention are compatible with the composition being safe and effective for the preventive and/or curative treatment of non-alcoholic fatty liver disease. More particularly, it has been determined that such quantities by weight guarantee synergistic activity between ingredients of the composition according to the invention.

Advantageously in a composition according to the invention, said at least a plant extract from the genus Desmodium as antioxidant is extracted from a plant selected from the group comprising Desmodium adscendens, Desmodium affine, Desmodium angustifolium, Desmodium arizonicum, Desmodium axillare, Desmodium barbatum, Desmodium batocaulon, Desmodium cajanifolium, Desmodium canadense, Desmodium canescens, Desmodium ciliare, Desmodium cinerascens, Desmodium cubense, Desmodium cuspidatum, Desmodium femaldii, Desmodium floridanum, Desmodium glabellum, Desmodium glabrum, Desmodium glutinosum, Desmodium grahamii, Desmodium gramineurn, Desmodium heterocarpon, Desmodium heterophyllum, Desmodium humifusum, Desmodium illinoense, Desmodium incanum, Desmodium intortum, Desmodium laevigatum, Desmodium lindheimeri, Desmodium lineaturn, Desmodium marilandicum, Desmodium metcalfei, Desmodium neomexicanum, Desmodium nudiflorum, Desmodium nuttallii, Desmodium obtusum, Desmodium ochroleucum, Desmodium paniculatum, Desmodium pauciflorum, Desmodium perplexum, Desmodium procumbens, Desmodium psilocarpum, Desmodium psilophyllum, Desmodium retinens, Desmodium rosei, Desmodium rotundifloium, Desmodium rotundifolium, Desmodium sandwicense, Desmodium scopulorum, Desmodium scorpiurus, Desmodium sericophyllum, Desmodium sessilifolium, Desmodium strictum, Desmodium tenuifolium, Desmodium tortuosum, Desmodium tritlorum, Desmodium tweedyi, Desmodium viridiflorum, Desmodium cuneatum, Desmodium elegans, Desmodium gangeticum, Desmodium gyrans, Desmodium micophyllum, Desmodium podocarpum, Desmodium styracifolium, Desmodium uncinatum and Desmodium wydlerianum, and mixtures thereof.

Preferably in a composition according to the invention, said at least a plant extract from the genus Desmodium as antioxidant is extracted from Desmodium adscendens.

Preferably in a composition according to the invention, said at least a flavolignan-type compound and/or said at least a compound comprising at least a flavonolignan-type molecule is selected from the group comprising silymarin (Silybum marianum), silybin, silibinin A, silibilin B and mixtures thereof.

Silymarin (Silybum marianum) contains at least seven different flavonolignan-type compounds, of which silibinin A and silibilin B are at the highest concentrations. In the sense of the invention, the term “one flavolignan-type compound” notably refers to silybin, silibinin A and silibilin B, and the term “compound comprising at least a flavonolignan-type molecule” notably refers to a whole extract of silymarin (Silybum marianum), i.e. a silymarin extract that contains all the flavolignans.

Preferably in a composition according to the invention, said at least a flavolignan-type compound and/or said at least a compound comprising at least a flavonolignan-type molecule is a naturally-occurring, synthetic or chemically modified compound. For example, a chemically modified and/or synthetic compound is silibinin dihydrogen disuccinate disodium.

Advantageously a composition according to the invention also contains at least an additional antioxidant selected from the group comprising superoxide dismutase (SOD), N-acetylcysteine, Vitamin C, Vitamin E, D-α-tocopherol, D-α-tocopheryl acetate, choline, zinc citrate, selenomethionione and glutathione, and mixtures thereof.

Preferentially said at least an additional antioxidant is superoxide dismutase (SOD).

Advantageously a composition according to the invention also contains at least a curcuma extract, in particular an extract of Curcuma longa (turmeric). It has been determined in the framework of this invention that adding at least a curcuma extract to a composition according to the invention comprising at least an antioxidant and at least a flavolignan-type and/or at least a compound comprising at least a flavonolignan-type molecule, further reinforces the effectiveness of the preventive/curative treatment of non-alcoholic fatty liver disease.

Preferably a composition according to the invention also contains at least a pharmaceutically acceptable excipient selected from the group comprising microcrystalline cellulose, magnesium stearate and mixtures thereof.

Preferentially a composition according to the invention is presented as a dietary supplement.

Advantageously a composition according to the invention is presented in a form that can be administered orally, e.g. as a tablet, capsule, pill, soluble powder, oral solution, oil-based suspension, effervescent tablet or soft capsule.

Other embodiments of a composition according to the invention are described in appended Claims.

Other features, details and advantages of the invention will emerge from the examples given below, without limitation and with reference to the appended figure.

### BRIEF DESORPTION OF THE DRAWINGS

FIG. 1 illustrates the rate of decrease (%) of ASAT, ALAT, gamma GT and fibrosis markers.

### EXAMPLES

1. Composition According to the Invention

Table 1 below is an example of a composition according to the invention in tablet form.

TABLE 1

| | | | Active substance | | % RDI* with two |
|---|---|---|---|---|---|
| Compound | Per tablet | | per 100 g | | tablets |
| Extract of | 100 | mg | 11.53 | g | ND*** |
| Curcuma longa | | | | | |
| Extract of | 100 | mg | 11.53 | g | ND |
| Silybum marianum** | | | | | |
| N-acetylcysteine | 100 | mg | 11.53 | g | ND |
| Choline | 90 | mg | 10.38 | g | ND |
| D-alpha-tocopheryl | 32.11 | mg | 3.7 | g | 250% |
| acetate | | | | | |
| Zinc citrate | 16.12 | mg | 1.86 | g | 100% |
| Extract of | 150 | mg | 17.25 | g | ND |
| Desmodium | | | | | |
| adscendens** | | | | | |
| Selenomethionine | 1 | mg | 0.12 | g | 18.16% |
| Magnesium stearate | 8.5 | mg | 1.02 | g | ND |

*RDI = recommended daily intake
**from the plant's aerial parts
***ND = not determined

2. Comparative Test

A comparative test was conducted on 25 patients of 18-65 years of age suffering from non-alcoholic fatty liver disease diagnosed in the previous 12 months. These patients were split between the four following groups:
Group 1 (n=6): control group given placebo in the form of tablets containing only talc;
Group 2 (n=6): given tablets of a composition known to the background art containing an extract of Silybum marianum and superoxide dismutase (SOD) as antioxidant;
Group 3 (n=6): given tablets of a composition according to the invention containing an extract of Silybum marianum and an extract of Desmodium adscendens as antioxidant;
Group 4 (n=7): given tablets of a composition according to the invention containing an extract of Silybum marianum, an extract of Desmodium adscendens as antioxidant and superoxide dismutase (SOD) as an additional antioxidant;

Before the beginning of treatment, blood was taken from every patient in each group and the plasma was stored at −80° C. until it could be assayed for cytokines and markers for fibrosis. These tests were carried out using enzyme-linked immunosorbent assay (ELISA) kits: IL-10, TNF-α, MMP-2, TGF-β (R&D Systems, Minneapolis, Minn., USA), hyaluronic acid (Echelon Biosciences, Salt Lake City, Utah, USA), procollagen I (Takara Bio, Otsu, Shiga, Japan), procollagen III (USCN Life Science, Wuhan, China), TIMP-1 (Invitrogen, Carlsbad, Calif., USA) and TIMP-2 (Chemicon International, Millipore, Billerica, Mass., USA).

The various tablets administered twice a day (one in the morning and one in the evening) to Groups 2, 3 and 4 contained the compounds in the quantities shown in Table 2.

TABLE 2

| | Group 2 | Group 3 | Group 4 |
|---|---|---|---|
| | Quantity | Quantity | Quantity |
| | (by weight | (by weight | (by weight |
| Compound | per tablet [mg]) | per tablet [mg]) | per tablet [mg]) |
| Extract of | 100 | 100 | 100 |
| Curcuma longa | | | |
| Extract of | 100 | 100 | 100 |
| Silybum marianum | | | |
| N-acetylcysteine | 100 | 100 | 100 |
| Choline | 90 | 90 | 90 |
| D-alpha-tocopheryl | 32.11 | 32.11 | 32.11 |
| acetate | | | |
| Zinc citrate | 16.12 | 16.12 | 16.12 |
| SOD | 10 | 0 | 10 |
| Selenomethionine | 1 | 1 | 1 |
| Magnesium stearate | 8.5 | 8.5 | 8.5 |
| Extract of | 0 | 150 | 150 |
| Desmodium | | | |
| adscendens | | | |

After six months of treatment, blood was taken from every patient in each group and the plasma was stored at −80° C. until it could be assayed for cytokines and markers for fibrosis (using the same ELISA kits as mentioned above).

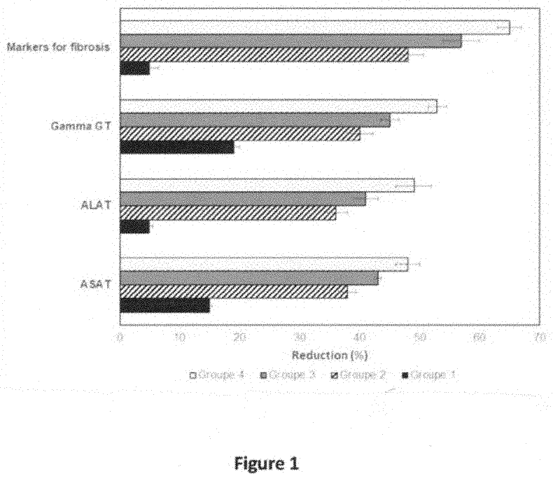

Results are presented in FIG. 1 which shows how the levels of AST, ALT, gamma GT and markers for fibrosis all dropped. The percentage drop is calculated as follows: [(reading aftertreatment−reading before treatment)/reading before treatment]*100.

As can be seen, a composition according to the invention (Group 3) induced significantly greater reductions in the levels of blood markers expressed by patients with non-alcoholic fatty liver disease, than those seen in the control group (Group 1) or the group given a composition known to the background art (Group 2). Moreover, the results obtained show that a composition according to the invention that also contains an additional antioxidant (in this case SOD) (Group 4) provides an even more statistically significant decrease in the levels of blood markers expressed by patients with non-alcoholic fatty liver disease.

This invention has been described with respect to specific embodiments which have purely illustrative value and should not be considered as limiting. In general terms, it will be obvious to those skilled in the art that this invention is not limited to the examples illustrated and/or described above.

Use of the words “comprise”, “contain”, “include” and any other variant, together with conjugated forms thereof, cannot in any way rule out the presence of ingredients other than those specifically mentioned.

Use of the indefinite article “a” or “an”, or the definite article “the” to qualify an item does not rule out the presence of more than one of said item.

1. Composition for use in the preventive and/or curative treatment of non-alcoholic fatty liver disease, in particular intended for use in the preventive and/or curative treatment of simple fatty liver disease and/or non-alcoholic fatty liver disease, said composition comprising:
at least a plant extract from the genus Desmodium as antioxidant, and
at least a flavonolignan-type compound and/or at least a compound comprising at least a flavonolignan-type molecule.

2. Composition for use according to claim 1, in which said at least a plant extract from the genus Desmodium as antioxidant is present at a concentration of 5-55% by weight compared to the total weight of the composition.

3. Composition for use according to claim 1, in which said at least a flavonolignan-type compound is present at a concentration of 5-90% by weight compared to the total weight of the composition.

4. Composition for use according to claim 1, in which said at least a compound comprising at least a flavonolignan-type molecule is present at a concentration of 5-90% by weight compared to the total weight of the composition.

5. Composition for use according to claim 1, in which said at least a plant extract from the genus Desmodium as antioxidant is extracted from a plant selected from the group comprising Desmodium adscendens, Desmodium affine, Desmodium angustifolium, Desmodium arizonicum, Desmodium axillare, Desmodium barbatum, Desmodium batocaulon, Desmodium cajanifolium, Desmodium canadense, Desmodium canescens, Desmodium ciliare, Desmodium cinerascens, Desmodium cubense, Desmodium cuspidatum, Desmodium femaldii, Desmodium floridanum, Desmodium glabellum, Desmodium glabrum, Desmodium glutinosum, Desmodium grahamii, Desmodium gramineum, Desmodium heterocarpon, Desmodium heterophyllum, Desmodium humifusum, Desmodium illinoense, Desmodium incanurn, Desmodium intortum, Desmodium laevigatum, Desmodium lindheimeri, Desmodium lineatum, Desmodium marilandicum, Desmodium metcalfei, Desmodium neomexicanum, Desmodium nudiflorum, Desmodium nuttallii, Desmodium obtusum, Desmodium ochroleucum, Desmodium paniculaturn, Desmodium pauciflorum, Desmodium perplexum, Desmodium procumbens, Desmodium psilocarpum, Desmodium psilophyllum, Desmodium retinens, Desmodium rosei, Desmodium rotundifloium, Desmodium rotundifolium, Desmodium sandwicense, Desmodium scopulorum, Desmodium scorpiurus, Desmodium sericophyllum, Desmodium sessilifolium, Desmodium strictum, Desmodium tenuifolium, Desmodium tortuosum, Desmodium triflorum, Desmodium tweedyi, Desmodium viridiflorum, Desmodium cuneaturn, Desmodium elegans, Desmodium gangeticum, Desmodium gyrans, Desmodium micophyllum, Desmodium podocarpum, Desmodium styracifolium, Desmodium uncinatum and Desmodium wydlerianum, and mixtures thereof.

6. Composition for use according to claim 1, in which said at least a flavolignan-type compound and/or said at least a compound comprising at least a flavonolignan-type molecule is selected from the group comprising silymarin (Silybum marianum), silybin, silibinin A, silibilin B and mixtures thereof.

7. Composition for use according to claim 1, in which said at least a flavolignan-type compound and/or said at least a compound comprising at least a flavonolignan-type molecule is a naturally-occurring, synthetic or chemically modified compound.

8. Composition for use according to claim 1, also containing at least an additional antioxidant selected from the group comprising superoxide dismutase (SOD), N-acetylcysteine, Vitamin C, Vitamin E, D-α-tocopherol, D-α-tocopheryl acetate, choline, zinc citrate, selenomethionione and glutathione, and mixtures thereof.

9. Composition for use according to claim 1, also containing at least a curcuma extract, in particular an extract of Curcuma longa (turmeric).

10. Composition for use according to claim 1, also containing at least a pharmaceutically acceptable excipient selected from the group comprising microcrystalline cellulose, magnesium stearate and mixtures thereof.

11. Composition for use according to claim 1, said composition being presented in the form of a dietary supplement.

12. Composition for use according to claim 1, said composition being presented in a form that can be administered orally.

13. Composition for use according to claim 2, in which said at least a plant extract from the genus Desmodium as antioxidant is present at a concentration of 10-30% by weight compared to the total weight of the composition

14. Composition for use according to claim 3, in which said at least a flavonolignan-type compound is present at a concentration of 15-30% by weight compared to the total weight of the composition

15. Composition for use according to claim 4, in which said at least a compound comprising at least a flavonolignan-type molecule is present at a concentration of 15-30% by weight compared to the total weight of the composition

16. Composition for use according to claim 12, said composition being presented as a tablet, capsule, pill, soluble powder, oral solution, oil-based suspension, effervescent tablet or soft capsule.
